(19) United States
(12) Patent Application Publication
(10) Pub. No.: US 2005/0272746 A1
(43) Pub. Date: Dec. 8, 2005

(54) Inactive isomer compositions for use as drug-resistance-reversal agents and in prophylactic treatment

(76) Inventor: Martin J. Smilkstein, Portland, OR (US)

Correspondence Address: KOLISCH HARTWELL, P.C., 520 S.W. YAMHILL STREET, SUITE 200, PORTLAND, OR, 97204, US

(21) Appl. No.: 11/145,467

(22) Filed: Jun. 2, 2005

Related U.S. Application Data
(60) Provisional application No. 60/576,752, filed on Jun. 2, 2004.

(51) Int. Cl. A61K 31/495; A61K 31/4172; A61K 31/137

(52) U.S. Cl. 514/255.04; 514/400; 514/649

(57) ABSTRACT

A method of using inactive isomer compositions as drug-resistance-reversal agents and in prophylactic treatment includes the steps of selecting an antihistaminically-inactive isomer of a preselected antihistamine, and making stereoselective use of the antihistaminically-inactive isomer for a clinical purposes. The making step includes choosing one of the following clinical purposes for which to make stereoselective use of the antihistaminically-inactive isomer: treatment of malaria, prophylaxis of malaria; and treatment of drug-resistant malignancies. The step of selecting an antihistaminically-inactive isomer involves preselecting an antihistamine from the group consisting of chlorpheniramine (−), btompheniramine (−), fluorpheniramine (−), pheniramine (−), bromodiphenhydramine, doxylamine, prophenpyridamine, chlorcyclizine, dimethindene (+), carbinoxamine (+), chlorphenoxamine, clemastine, orphenadrine, hydroxyzine, meclizine, and buclizine. Various inactive isomer compositions are disclosed for the above-described clinical purposes.

### CROSS-REFERENCE TO RELATED APPLICATIONS

This application claims priority to U.S. Provisional Patent Application Ser. No. 60/576,752, filed Jun. 2, 2004 and entitled “INACTIVE ISOMER COMPOSITIONS FOR USE AS DRUG-RESISTANCE-REVERSAL AGENTS AND IN PROPHYLACTIC TREATMENT”, the disclosure of which is incorporated herein by reference in its entirety for all purposes.

### BACKGROUND

It is well known that some chemical compounds containing the same constituents can exist in more than one arrangement, called isomers. When compounds have identical constituents, and identical bonds, but differ only in spatial arrangement, these are referred to as stereoisomers. Stereoisomers of a given chemical compound, despite sharing identical chemical composition, often have very different actions when placed in biologic systems. This property is sometimes referred to as stereoselectivity, and forms the basis for both therapeutic and research selection of certain stereoisomers of a compound for specific purposes.

In the case of a chemical compound used as a drug, selection of one drug isomer over another may be to increase potency, decrease toxicity, or both.

Since World War II, the drug chloroquine has been the most important treatment for the disease malaria. Unfortunately, the parasite Plasmodium that causes malaria, including the species most lethal to humans, Plasmodium falciparum, has become progressively resistant to chloroquine. As a result, chloroquine treatment failures and in vitro chloroquine resistance have been documented throughout the majority of the world where malaria is found. Similarly, resistance to other antimalarial drugs including quinine, mefloquine, halofantrine, pyrimethamine-sulfadoxime, amiodaquine, atovaquone and others has also been noted.

For decades, since the first chloroquine resistant malaria was documented, extensive research has been done to both understand the mechanism(s) by which drug resistance develops and to find new ways to effectively treat drug-resistant malaria. In addition to seeking new drugs to kill the parasite, efforts have been made to reverse chloroquine resistance and render resistant parasites sensitive to chloroquine. Many drugs in diverse structural and functional classes have been found to partly or completely restore chloroquine sensitivity to P. falciparum in vitro, but few have been tried in vivo, and very few have reached clinical trials. The most promising thus far has been chlorpheniramine, which when co-administered with chloroquine improved outcomes when compared to chloroquine alone.

Although the combination of chlorpheniramine with chloroquine was superior, small numbers of treatment failures and drowsiness was consistently found after chlorpheniramine dosing. These trials suggested that either higher chlorpheniramine dosing, longer courses of therapy, or addition of a third drug would each be expected to succeed, but each of these plans is seriously limited by safety, compliance likelihood, cost or simplicity concerns. For this reason, and with the advent of alternative new drugs, the investigation of chloroquine-resistance reversal for clinical use has largely disappeared.

Chlorpheniramine, like all traditional antihistamines used to treat allergic symptoms, exerts its desired anti-allergy effect by interfering with the access of histamine to one of its sites of action, the so-called H1 receptor. Unlike newer H1 receptor blockers, that cause little in the way of sedation, traditional (a.k.a. first-generation), H1 receptor blockers like chlorpheniramine consistently cause drowsiness, apparently mediated by H1 receptor blockade in the central nervous system.

It has been known for decades that the chlorpheniramine exists in two forms, one known as d or (+) chlorpheniramine, and the other as l or (−) chlorpheniramine. The H1 blockade, and thus the desired antihistamine effect, results from the d or (+) chlorpheniramine which is 50-100 times more potent than the l or (−) form in this action. It is also the d or (+) form which causes sedation.

### SUMMARY OF THE INVENTION

The inventions involve a method and related compositions that make stereoselective use of the antihistaminically-inactive isomers of traditional antihistamines for the following clinical purposes:
1. Treatment of malaria (most importantly drug-resistant malaria), in combination with other anti-malarial drugs (including but not limited to chloroquine, quinine, halofantrine, and mefloquine);
2. Prophylaxis of malaria; and
3. Treatment of drug-resistant malignancies.

The ability of traditional (so-called first generation) antihistamines to reverse malaria drug-resistance in vitro is well-established. When used in clinical trials, a major limitation of antihistamines has been the drowsiness or sedation they cause. For some antihistamines that exist as more than one stereoisomer, it is also well-established that both the antihistamine effect and the sedation result from the same isomer. As part of malaria treatment, the most studied of the antihistamines is chlorpheniramine, which exists as either the dextrorotatory (a.k.a., dextro, d, or (+)) isomer or the levorotatory (a.k.a., levo, l, or (−)) isomer, and is generally used as a mixture, symbolized as (+/−) chlorpheniramine. Only the (+) isomer is an active antihistamine, and the same isomer causes the sedative side effect. The inventions include surprise result that the, in vitro, (−)chlorpheniramine is effective at reversing drug-resistance in malaria parasites. By using the non-sedating isomer of antihistamines proven to reverse drug-resistance, higher dosing and thus greater efficacy might be achieved.

The inventions have established that the antihistaminically inactive (−) chlorpheniramine reverses both chloroquine resistance and quinine resistance in Plasmodium falciparum, the parasite causing the most lethal form of malaria. The inventions also include tests for a related antihistamine, brompheniramine, with chloroquine, showing the same result. Those tests also show that the racemic (+/−) mixtures of other antihistamines, distinct from those already described in the literature, are also effective resistance reversers. The inventions therefore include a treatment method that makes stereoselective use of any antihistamine which exists as stereoisomers and which is effective as a drug-resistance reversal agent.

The identity of the antihistamine-active form has been clearly identified for some antihistamines, but not for others, so it is only possible to specifically name a few from a list of those with more than one stereoisomer. The following list includes several of the antihistamines covered by the inventions and, if known, the antihistamine-inactive form. It should be noted that many other, less well-known antihistamines exist that are not listed but are within the scope of the inventions:

Partial list of steroisomeric antihistamines (antihistamine-inactive isomer, if known)
1. chlorpheniramine (−)
2. brompheniramine (−)
3. fluorpheniramine (−)
4. pheniramine (−)
5. bromodiphenhydramine
6. doxylamine
7. prophenpyridamine
8. chlorcyclizine
9. dimethindene (+)
10. carbinoxamine (+)
11. chlorphenoxamine
12. clemastine
13. orphenadrine
14. hydroxyzine
15. meclizine
16. buclizine

The inventions include the use of antihistamines (specifically (−) chlorpheniramine) as part of combination therapy against drug-resistant malaria (specifically chloroquine-resistant P. falciparum), and the inventions extend to the related pheniramines (brompheniramine, fluorpheniramine, pheniramine), the other steroisomeric antihistamines, to other antimalarial drug-resistance (e.g., quinine, halofantrine, mefloquine) and to other forms of malaria (e.g., P. vivax, P. ovale, P. malariae). In addition, a small number of studies has shown that antihistamines can be effective prophylaxis in a rodent malaria model. Because of the evident similarities in mechanisms and actions between the antihistamines, the invention also covers the application of these inactive antihistamine isomers to use as prophylactic treatment.

The inventions may also be used to treat malignancy based on the observation of important similarities between multi-drug-resistant cancer cells and drug-resistant malaria, both in mechanism and in the drugs that reverse the phenomenon. The same agents that reverse drug-resistance in malaria should be similarly effective in some models of cancer drug-resistance.

In addition to antihistamines, other classes of drugs show the same resistance-reversal effect, and are also part of the inventions. The common names used to describe the other classes of drugs included in the inventions are tricyclic antidepressants and phenothiazine antipsychotics. They are potent in vitro drug-resistance-reversal agents against malaria, but cause dose-limiting adverse effects in humans. Unlike the antihistamines which have a single characteristic upon which to base selection of an isomer under the inventions, these agents have several adverse actions. The inventions propose identifying that any or all of these adverse effects are selectively caused by one isomer, with the other isomer being a potent resistance-reversal agent.

An abstract of test results achieved according to the inventions follows:

Reversing Chloroquine Resistance: A Stereoselective Approach to a Stereo-Indifferent Target.

In clinical trials, co-administration of the antihistamine chlorpheniramine (CP) with chloroquine (CQ) has shown promise against CQ-resistant P. falciparum infection. Shortcomings of this approach have included treatment failures with short course (3 day) regimens and dose-related sedation from CP. Therefore, it appears likely that longer courses of therapy, higher CP dosing or the addition of another antimalarial agent might achieve improved clinical efficacy, but each of these approaches would limit the feasibility, safety or advantage of using a resistance-reversal strategy. We therefore performed in vitro studies of CP enantiomers to determine if stereoselective CP use offers additional benefit that might translate to clinical feasibility. We compared the potency of the currently available (+) and (+/−) forms of CP to reverse in vitro CQ resistance of P. falciparum strains W2 and 7G8. Although only the (+)CP isomer is an effective antihistamine, it is the racemic (+/−)CP mixture that is generally used clinically, and that has been previously studied in combination with CQ. The EC₅₀ of CQ in each strain was determined several times in the absence of CP and in the presence of various concentrations of (+)CP or (+/−)CP using a 72 hour microplate growth assay. Results were determined by SybrGreen I-based fluorescence assay. Equimolar (+/−)CP was at least equivalent to (+)CP in decreasing CQ EC₅₀ in all assays, indicating that both the (+) and (−) enantiomers are active as CQ-resistance reversal agents. Although the CP site of CQ resistance reversal appears to be stereo-indifferent, these results still suggest that a stereoselective approach deserves study; not to achieve greater potency, but to avoid toxicity. Because it has been shown that (−)CP does not cause sedation, and thus may prove safe at far higher doses than possible with either (+) or (+/−)CP, this demonstration of in vitro CQ-resistance reversal efficacy suggests that CQ and (−)CP in combination might reach a clinically useful threshold.

Another description of the inventions follows:

(−)Chlorpheniramine to Reverse Chloroquine Resistance in P. falciparum: A Stereoselective Approach to a Stereoindifferent Target

In tandem with new drug development, innovative exploration of existing pharmaceuticals is a valuable weapon in the global fight against malaria and other neglected diseases. Using a novel approach, we propose to build upon earlier work showing the effectiveness of antihistamines (drugs widely used in the treatment of allergic symptoms) to reverse chloroquine resistance. It is our contention that a rapidly available, clinically effective drug combination can result from this investigation. There are several reasons to again elevate the study of chloroquine resistance reversal from a mechanistic probe and research tool to serious clinical consideration. Unlike new drugs, chloroquine is widely and immediately available, inexpensive and its dosing and safety profile is extensively understood. As a result, for most of the malaria-affected world, restoration of chloroquine effectiveness would be the single most powerful possible antimalarial drug advance. Resistance “reversal” is possible because, unlike resistance to other drugs (e.g.,pyrimethamine-sulfadoxine) from mutations which irreversibly prevent drug action, the mechanism of action of chloroquine persists even in resistant parasites. If enough chloroquine gains and retains access to its site of action, it is still effective, even in chloroquine-resistant parasites. This fact, clearly demonstrated in vitro, in vivo and in clinical trials leaves no doubt that the concept is sound; the challenge is to accomplish chloroquine resistance reversal in a manner that is effective, safe, inexpensive and feasible.

The observation that multidrug resistance in cancer cells could be reversed by the addition of other drugs led Martin and colleagues to investigate whether an analogous result could be produced in drug-resistant malaria. Their finding that verapamil partially restored chloroquine sensitivity to resistant P. falciparum led to subsequent discovery of a long list of diverse compounds, in several structural classes, with similar results. While most are experimental drugs only, several commonly used pharmaceutical products have shown antimalarial drug resistance reversal properties.

Some, like verapamil, are not candidates for clinical use for this purpose because of their known toxicity at concentrations needed to produce the desired effect. The tricyclic antidepressant, desipramine, reversed chloroquine resistance in vitro and in a primate animal trial, but was ineffective in clinical trial. Several antihistamine drugs are effective in vitro and in rodent studies, and a few have reached clinical trials. The most promising has been chlorpheniramine. In addition to its demonstrated effectiveness in vitro and in rodent malaria models, chlorpheniramine-chloroquine combination therapy has resulted in improved parasitological and clinical cure rates compared with chloroquine alone, and has been comparable to other combination drug protocols.

Despite the success of chlorpheniramine-chloroquine combination therapy, several shortcomings are evident. Chlorpheniramine, like all older antihistamines, consistently causes drowsiness which interferes with outpatient compliance and limits the dose which can be used. Secondly, along with a 3-day course of chloroquine, it was suggested that chlorpheniramine for 7 days, or the addition of a third drug was needed to safely treat resistant infections; another serious challenge to real-world compliance. Lastly, although the combination was far superior to chloroquine alone, there were small numbers of treatment failures in these studies. Taken in total, the evidence indicates that combination therapy with chlorpheniramine and chloroquine is close, but not close enough to being a weapon against chloroquine resistant malaria.

The basis for our proposal to re-investigate chlorpheniramine and other antihistamines is the exploitation of the well-known concept of stereoselectivity. Stereoselectivity, that is, a differential effect between mirror-image isomers, is common in biological systems. Therefore, one isomer of a given compound may be potent in producing an effect, and the other isomer, although chemically identical, may not be. If the desired effect of a drug and its undesired adverse effects are mediated by different isomers, this affords the chance to isolate and use only the desired isomer for therapy. This principle is commonly used in pharmaceutical selection, and our studies indicate that such an approach would be successful with chlorpheniramine.

The antihistamine effect of chlorpheniramine is mediated by its (+) isomer (50-100 times the potency of the (−) isomer), but so is the sedation it causes. Volunteer studies and neurophysiologic models show that the (−) isomer causes neither drowsiness or its neurochemical correlates. In addition, (−)chlorpheniramine is less bound by serum proteins and changes cardiac electrophysiology only at in vitro concentrations far above those needed to restore chloroquine sensitivity. Because nearly all commercially available chlorpheniramine formulations (including that used in the aforementioned studies) are racemic mixtures of (+/−)chlorpheniramine, there is already extensive experience with both isomers in clinical use, misuse and in overdose. All indications are that (−)chlorpheniramine would be expected to be safe at doses several-fold higher than either the (+) or (+/−) forms.

To determine whether or not (−)chlorpheniramine is effective in chloroquine resistance reversal, we compared equimolar (+) and (+/−)chlorpheniramine. This indirect assessment was required because only the antihistaminically-active (+) and (+/−) forms are available. In both the Indochina-derived strain W2 and the South American strain 7G8, for both chloroquine and quinine, the (+/−)chlorpheniramine mixture was equivalent or slightly superior to the (+) isomer, in reversing drug resistance, indicating that the (−) isomer of chlorpheniramine must be active. If (−)chlorpheniramine is equipotent to (+)chlorpheniramine as a resistance reversal agent, although this implies that the target is stereoindifferent, a stereoselective approach would be expected to reduce adverse effects, allow higher dosing and thus allow combination therapy to reach an acceptable threshold of effectiveness.

We therefore propose to isolate, purify and test (−)chlorpheniramine with chloroquine in vitro against new and old world chloroquine-resistant P. falciparum strains, and subsequently to test the combination in rodent models. In vitro testing will also include a similar analysis of brompheniramine, flupheniramine and the non-halogenated pheniramine, all of which we have found to be chloroquine resistance reversal agents, and all of which exist as both (+) and (−) isomers. Depending on these results, consideration will be given to a similar analysis of the short list of remaining antihistamines which also exist as stereoisomers.

The specific embodiments of the above-disclosed compositions and methods are not to be considered in a limiting sense as numerous variations are possible. The subject matter of this disclosure includes all novel and non-obvious combinations and subcombinations of the various features, elements, functions and/or properties disclosed herein. No single feature, function, element or property of the disclosed embodiments is essential. The following claims define certain combinations and subcombinations which are regarded as novel and non-obvious. Other combinations and subcombinations of features, functions, elements and/or properties may be claimed through amendment of the present claims or presentation of new claims in this or a related application. Such claims, whether they are different, broader, narrower or equal in scope to the original claims, are also regarded as included within the subject matter of the disclosure.

1. A method of using inactive isomer compositions as drug-resistance-reversal agents and in prophylactic treatment, comprising:
selecting an antihistaminically-inactive isomer of a preselected antihistamine; and
making stereoselective use of the antihistaminically-inactive isomer for a clinical purposes.

2. The method of claim 1 wherein the making step includes choosing one of the following clinical purposes for which to make stereoselective use of the antihistaminically-inactive isomer: treatment of malaria, prophylaxis of malaria; and treatment of drug-resistant malignancies.

3. The method of claim 1 wherein the selecting an antihistaminically-inactive isomer involves preselecting an antihistamine from the group consisting of chlorpheniramine (−), brompheniramine (−), fluorpheniramine (−), pheniramine (−), bromodiphenhydramine, doxylamine, prophenpyridamine, chlorcyclizine, dimethindene (+), carbinoxamine (+), chlorphenoxamine, clemastine, orphenadrine, hydroxyzine, meclizine, and buclizine.
